(19) United States
(12) Patent Application Publication
(10) Pub. No.: US 2026/0279595 A1
(43) Pub. Date: Sep. 17, 2026

(54) SYSTEM AND METHOD FOR REMOTE DATA COLLECTION AND OUTCOME PREDICTION

(71) Applicant: UTI Limited Partnership, Calgary (CA)

(72) Inventors: Aaron Alexander Phillips, Calgary (CA); Kelly Anne Kaiser, Calgary (CA); Julien-Yoann Rimok, Calgary (CA)

(21) Appl. No.: 19/672,218

(22) Filed: May 8, 2026

Related U.S. Application Data
(63) Continuation of application No. PCT/CA2024/051461, filed on Nov. 5, 2024.
(60) Provisional application No. 63/548,019, filed on Nov. 10, 2023.

(51) Int. Cl. G16H 50/30; G16H 50/20

(52) U.S. Cl. CPC G16H 50/30; G16H 50/20

(57) ABSTRACT

Provided are computer-implemented systems and methods for generating a health outcome prediction, including: receiving user data, the user data comprising passively collected data by a passive data collection monitoring device associated with the user; self-reported health metrics associated with the user; receiving clinical health outcome data, the clinical health outcome data comprising at least one health outcome based on clinical data; extracting a plurality of variables from the user data; providing the plurality of variables and the clinical health outcome data as inputs to at least one machine learning model to identify at least one selected variable from the plurality of variables, having an associated predictive value to the clinical health outcome data; generating based on the at least one selected variable, an individualized prediction algorithm for generating the health outcome prediction; applying the individualized prediction algorithm to the user data to generate the health outcome prediction.

### CROSS-REFERENCE TO RELATED APPLICATION

The application is a continuation of International Application No. PCT/CA2024/051461, filed Nov. 5, 2024, which claims priority from U.S. Provisional Patent Application No. 63/548,019, filed on Nov. 10, 2023, the entire contents of which are hereby incorporated by reference for all purposes.

### FIELD

The described embodiments relate to systems, devices and methods for generating a health outcome prediction for a user and more specifically systems, devices and methods for generating an individualized health outcome prediction using a combination of passively collected data via wearable passive data collection monitors, and self-reported health metrics in combination with clinical health outcome data, and application usage metric data.

### BACKGROUND

There is a need for improved real-world data collection platforms focused on health functions, particularly for subjects living with ongoing or chronic conditions and compromised health. Real-world data collection is increasingly advantageous for executing clinical studies due to reduced participant burden and cost, flexibility of design, and increased external validity of findings. Ongoing or chronic conditions and compromised health may include many aspects, such as autonomic function, and are not often measured in clinical research despite being a significant source of health care costs, and a top patient priority.

Conventional attempts to collect real-world evidence of ongoing or chronic conditions and compromised health (for example, self-reported health metrics such as qualitative questionnaire data) have relied on email transfer of burdensome questionnaires with low compliance.

Inclusion of real-world data collection in addition to traditional clinical research approaches promises substantial cost reductions. By leveraging readily available technology and remote monitoring, researchers can minimize overhead expenses associated with clinical study sites, transportation, and on-site data collection. This cost-efficiency can make clinical studies more accessible, therefore, inclusive of more diverse patient populations, and affordable for both researchers and participants, including those with limited access to participation at study sites.

Traditional clinical studies often follow rigid protocols, which may not adequately accommodate the diverse needs and challenges of individuals living with a variety of health concerns. As part of the studies, researchers often rely on participants to complete lengthy questionnaires in person, or sent to them in over e-mail. In contrast, remote data collection allows for tailored approaches that consider the unique circumstances and abilities of participants. This adaptability enhances patient engagement, reduces the patient burden of participation, and ultimately improves patient retention which are critical factors in the success of clinical studies/trials.

Real-world data also allows for large and diverse sample sizes and rapid digitization of physiological, behavioral, and symptom changes of end-users. This large sample and rapid digitization permit for individualized and real-time prediction of health outcomes risk, which would enhance patient awareness, health care delivery and research.

Autonomic functions, which regulate many vital biological processes like heart rate, blood pressure, sleep, and digestion, play a pivotal role in overall health and should be a key consideration in clinical studies. Neglecting to monitor autonomic function in the real-world setting is a missed opportunity for capturing and understanding the comprehensive clinical characteristics of patients participating in clinical studies, resulting in incomplete data, while also failing to address the heterogeneous health experience of people living with compromised health. A significant source of healthcare costs are associated with autonomic dysfunction. Individuals with a variety of clinical conditions face autonomic dysfunction-related health issues, making it a top patient and clinical research priority.

Most attempts to collect self-report health metric data, in combination with real-world evidence have relied on email transfer and burdensome questionnaires with low compliance, resulting in poor data to accurately, and efficiently, predict a health outcome for an end-user with chronic conditions and compromised health.

Due to these restrictions on the collection of health metric data, conventional approaches to predicting health outcomes are based on generalized models and predictions and do not offer individualized approaches to prediction.

Therefore, there exists a need for a process to collect real-world data (passively and actively) in combination with clinical study data to monitor health outcomes and be able to generate accurate individualized health outcome predictions, for those living with compromised health conditions.

### SUMMARY

Provided herein is a real-world data collection system and device that focuses on chronic conditions and compromised health, for example, autonomic functions. The system and device may be paired with a method for individualized prediction of health outcomes-empowering end-users enhanced knowledge of their health status, and allowing researchers and clinicians to bridge a significant gap between healthcare and research. This system and method enhances the external validity of findings and ensures that clinical study results are applicable to a broader population of patients that would not typically be included in traditional homogenous clinical study/trial populations. Moreover, this platform can empower individuals living with compromised health to have rapid and personalized awareness of their health event risk, and actively participate in research, and the development of their individual health knowledge. The most promising feature of this platform is the individualized health outcome predictions using passively collected data (physiological and behavioral), and self-reported health metrics collected in the real-world setting.

Advantages of the present platform include the ability to predict health outcomes using data collected in real-world and clinical study settings including unique features for wheelchair users, simple delivery of abbreviated questions, passive data collection using wearable technology, and integration with digitized clinical health records.

In a first aspect, there is provided a computer-implemented method for generating a health outcome prediction for a user, the method comprising: receiving, at a processor, user data associated with the user, the user data comprising monitoring data collected by a passive data collection monitoring device in communication with the processor, the passive data collection monitoring device associated with the user; receiving, at the processor, clinical health outcome data, the clinical health outcome data comprising at least one health outcome based on clinical data associated with at least one individual; extracting, at the processor, a plurality of features from the user data; providing, at the processor, the plurality of features and the clinical health outcome data as inputs to at least one machine learning model to identify at least one selected feature from the plurality of features, the at least one selected feature having an associated predictive value to the clinical health outcome data for the user; generating, at the processor, based on the at least one selected feature, an individualized prediction algorithm for generating the health outcome prediction for the user, the individualized prediction algorithm being individualized to the user; applying, at the processor, the individualized prediction algorithm to the user data to generate the health outcome prediction of the user; and outputting the health outcome prediction.

In one or more embodiments, the method may further comprise: prior to extracting, at the processor, the plurality of features from the user data, generating, at the processor, one or more features from the received user data, wherein the user data comprises the received user data and the one or more features generated from the received user data; and generating, at the processor, one or more features from the received clinical health outcome data, wherein the clinical health outcome data comprises the received clinical health outcome data and the one or more features generated from the received clinical health outcome data.

In one or more embodiments, the user data may further comprise self-reported health metric data, the self-reported health metric data comprising at least one user-provided answer to at least one prompt.

In one or more embodiments, the method may further comprise: providing, at a user input device in communication with the processor, the at least one prompt; and receiving, at the user input device, the at least one user-provided answer corresponding to the at least one prompt.

In one or more embodiments, the user input device may comprise the passive data collection monitoring device.

In one or more embodiments, the at least one user-provided answer may provide data relating to at least one selected from the group of self-reported health metric data of the user, life event data of the user, appointment data of the user, test result data of the user, medication data of the user and quality of life data of the user.

In one or more embodiments, the passive data collection monitoring device may comprise at least one selected from the group of a wearable device, a smartwatch, a smartphone, an accelerometer, a gyroscope, an electrocardiogram sensor, an electromyogram sensor, an arterial pulse sensor, a temperature sensor, a blood oxygen saturation sensor, a tissue carbon dioxide sensor, and an electrical impedance monitor.

In one or more embodiments, the clinical health outcome data may be received from at least one database associated with a patient registry, disease registry, healthcare database, system, or provider.

In one or more embodiments, the at least one database may comprise data associated with a plurality of individuals, the data corresponding to at least one selected from the group of patient registry, disease registry, healthcare system or provider admissions data, surgery data, intervention data, diagnostic data, medication data, and specialist care data.

In one or more embodiments, the clinical health outcome data may further comprise data generated from the data of the at least one database, the generated data corresponding to at least one selected from the group of organ health data, organ function data, mental health data, cognitive function data, sleep data, mood data, pain data, sexual function data and mobility data.

In one or more embodiments, the generated data may be associated with the user.

In one or more embodiments, the plurality of features may comprise a plurality of variables corresponding to at least one selected from the group of physical activity data of the user, body composition data of the user, movement data of the user, sleep data of the user, organ health data of the user, organ function data of the user, sexual health data of the user, sexual function data of the user, mental health data of the user, cognitive function data of the user, productivity data of the user, mood data of the user, pain data of the user, symptoms data of the user, nutrition data of the user, medication data of the user, self-care data of the user, and wellness data of the user.

In one or more embodiments, the identifying at least one selected feature from the plurality of features may comprise mapping, at the processor, a plurality of variables and the clinical health outcome data.

In one or more embodiments, the mapping, at the processor, the plurality of variables and the clinical health outcome data may comprise performing, at the processor, at least one machine learning modelling technique on the plurality of variables and the clinical health outcome data.

In one or more embodiments, the at least one machine learning technique may be a dimensionality reduction based on the plurality of variables and the clinical health outcome data.

In one or more embodiments, the generating the individualized prediction algorithm may comprise performing machine learning based on the at least one selected variable.

In one or more embodiments, the implementing the machine learning model based on the at least one selected feature may comprise: determining a similarity metric for the at least one selected variable based on a desired clinical health outcome; and selecting at least one of the at least one selected variable based on the corresponding similarity metric.

In one or more embodiments, the method may further comprise: providing, in the memory, a population-level data set; extracting a set of population-level or sub-population features from the population-level data set at the processor; and wherein the plurality of features comprises the extracted set of population-level or sub-population features.

In one or more embodiments, the method may further comprise applying, at the processor, at least one goal function to the individualized prediction algorithm to improve goodness-of-fit of the individualized prediction algorithm.

In one or more embodiments, the applying, at the processor, the at least one goal function to the individualized prediction algorithm may occur iteratively.

In one or more embodiments, the at least one goal function may be at least one selected from the group of a mean squared error, an absolute value error, a root mean squared error value, an adjusted R2 value, an Akaike information criterion value, an absolute error value, a cross-entropy error value, a p-value based on a f-statistic value, or any other commonly used functions in model.

In one or more embodiments, the goal function may be optimized through minimization or maximization, depending on the goal function.

In one or more embodiments, the root mean squared error value may be minimized.

In one or more embodiments, the outputting the health outcome prediction may comprise outputting the health outcome prediction at a user software application on a user device.

In one or more embodiments, the user device may comprise the passive data collection monitoring device.

In one or more embodiments, the outputting the health outcome prediction may comprise outputting the health outcome prediction at a clinical software application on a clinical device.

In a second aspect, there is provided a system for generating a health prediction outcome for a user, the system comprising: a memory comprising: at least one machine learning model; a database comprising clinical health outcome data; a processor in communication with the memory, the processor configured to: provide, in the memory, user data associated with the user, the user data comprising passive data collection monitoring data, the monitor data collected by a passive data collection monitoring device in communication with the processor, the passive data collection monitoring device associated with the user; receive, from the database, the clinical health outcome data, the clinical health outcome data comprising at least one health outcome based on clinical data associated with at least one individual; extract a plurality of features from the user data; provide the plurality of features and the clinical health outcome data as inputs to at least one machine learning model to identify at least one selected feature from the plurality of features, the at least one selected feature having an associated predictive value to the clinical health outcome data for the user; generate, based on the at least one selected feature, an individualized prediction algorithm for generating the health outcome prediction for the user, the individualized prediction algorithm being individualized to the user; apply the individualized prediction algorithm to the user data to generate the health outcome prediction of the user; and output the health outcome prediction.

In one or more embodiments, the processor may be further configured to: prior to the processor being configured to extract a plurality of features from the user data, generate one or more features from the provided user data, wherein the user data comprises the provided user data and the one or more features generated from the provided user data; and generate one or more features from the received clinical health outcome data, wherein the clinical health outcome data comprises the received clinical health outcome data and the one or more features generated from the received clinical health outcome data.

In one or more embodiments, the processor may be further configured to store the user data in the database.

In one or more embodiments, the user data may further comprise self-reported health metric data, the self-reported health metric data comprising at least one user-provided answer to at least one prompt.

In one or more embodiments, the system may further comprise: a user input device in communication with the processor, the user input device configured to provide the at least one prompt and receive the at least one user-provided answer corresponding to the at least one prompt.

In one or more embodiments, the user input device may comprise the passive data collection monitoring device.

In one or more embodiments, the at least user-provided answer may provide data relating to at least one selected from the group of symptom data of the user, life event data of the user, appointment data of the user, test result data of the user, medication data of the user and quality of life data of the user.

In one or more embodiments, the passive data collection monitoring device may comprise at least one selected from the group of a wearable device, a smartwatch, a smartphone, an accelerometer, a gyroscope, an electrocardiogram sensor, an electromyogram sensor, an arterial pulse sensor, a temperature sensor, a blood oxygen saturation sensor, a tissue carbon dioxide sensor, and an electrical impedance sensor.

In one or more embodiments, the system may comprise a network interface in communication with the processor, the network interface configured to receive the clinical health outcome data from a second database associated with a healthcare provider.

In one or more embodiments, the second database may comprise data associated with a plurality of individuals, the data corresponding to at least one selected from the group of healthcare provider admissions data, surgery data, intervention data, diagnostic data, medication data and specialist care data.

In one or more embodiments, the clinical health outcome data may comprise data generated from the data of the second database, the generated data corresponding to at least one selected from the group of organ health data, organ function data, mental health data, cognitive function data, sleep data, mood data, pain data, sexual function data, nutrition data, and mobility data.

In one or more embodiments, generated data may be associated with the user.

In one or more embodiments, the plurality of features may comprise a plurality of variables corresponding to at least one selected from the group of a physical activity data of the user, body composition data of the user, movement data of the user, sleep data of the user, organ health data of the user, organ function data of the user, sexual health data of the user, sexual function data of the user, mental health data of the user, cognitive function data of the user, productivity data of the user, mood data of the user, pain data of the user, symptoms data of the user, nutrition data of the user, medication data of the user, self-care data of the user, and wellness data of the user.

In one or more embodiments, the processor being configured to identify at least one selected feature from the plurality of features may comprise the processor being configured to map the plurality of variables and the clinical health outcome data.

In one or more embodiments, the processor being configured to map the plurality of variables and the clinical health outcome data may comprise the processor being configured to perform at least one machine learning modelling technique on the plurality of variables and the clinical health outcome data.

In one or more embodiments, the at least one machine learning model may be a dimensionality reduction based on the plurality of features and the clinical health outcome data.

In one or more embodiments, the processor being configured to generate the individualized prediction algorithm may comprise the processor being configured to perform machine learning based on the at least one selected variable.

In one or more embodiments, the processor being configured to perform the dimensionality reduction based on the at least on selected variable may comprise the processor being configured to: determine a similarity metric for the at least one selected variable based on a desired clinical health outcome; and select at least one of the at least one selected variable based on the corresponding similarity metric.

In one or more embodiments, the memory may comprise: providing, in the memory, a population-level data set; and the processor may be further configured to: extract a set of population- or sub-population level features from the population-level data set; and wherein the plurality of features comprises the extracted set of population-level or sub-population-level features.

In one or more embodiments, the processor may be further configured to apply at least one goal function to the individualized prediction algorithm to improve goodness-of-fit of the individualized prediction algorithm.

In one or more embodiments, processor may be further configured to iteratively apply the at least one goal function to the individualized prediction algorithm.

In one or more embodiments, the at least one goal function may be selected from the group of a mean squared error, an absolute value error, a root mean squared error value, an adjusted R2 value, an Akaike information criterion value, an absolute error value, a cross-entropy error value, a p-value based on a f-statistic value, or any other commonly used functions in model optimization.

In one or more embodiments, the goal function may be optimized through minimization or maximization, depending on the goal function.

In one or more embodiments, the root mean squared error value may be minimized.

In one or more embodiments, the processor may be further configured output the health outcome prediction at a user software application on a user device.

In one or more embodiments, the user device may comprise the passive data collection monitoring device.

In one or more embodiments, the processor may be further configured to output the health outcome prediction at a clinical software application on a clinical device.

### BRIEF DESCRIPTION OF THE DRAWINGS

A preferred embodiment of the present invention will now be described in detail with reference to the diagrams, in which:

FIG. 1 shows a system diagram in accordance with one or more embodiments.

FIG. 2 shows a device diagram in accordance with one or more embodiments.

FIG. 3 shows another device diagram in accordance with one or more embodiments.

FIGS. 4A, 4B, 4C, and 4D show user interface diagrams in accordance with one or more embodiments.

FIGS. 5A and 5B show another example of a user interface diagram in accordance with one or more embodiments.

FIG. 6 shows an example user interaction workflow in accordance with one or more embodiments.

FIG. 7 shows a flowchart in accordance with one or more embodiments.

FIGS. 8A and 8B show examples of individualized prediction algorithms for different subjects in accordance with one or more embodiments.

FIG. 9 shows a method diagram in accordance with one or more embodiments.

FIG. 10 shows another example of an individualized prediction algorithm in accordance with one or more embodiments.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

Various apparatuses or methods will be described below to provide an example of the claimed subject matter. No example described below limits any claimed subject matter and any claimed subject matter may cover methods or apparatuses that differ from those described below. The claimed subject matter is not limited to apparatuses or methods having all the features of any one apparatus or methods described below or to features common to multiple or all the apparatuses or methods described below. It is possible that an apparatus or methods described below is not an example that is recited in any claimed subject matter. Any subject matter disclosed in an apparatus or methods described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It should also be noted that the terms “coupled” or “coupling” as used herein can have several different meanings depending on the context in which these terms are used. For example, the terms “coupled” or “coupling” can have a mechanical, electrical or communicative connotation. For example, as used herein, the terms “coupled” or “coupling” can indicate that two elements or devices can be directly connected to one another or connected to one another through one or more intermediate elements or devices via an electrical element, electrical signal or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as "substantially", "about" and "approximately" as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term "about" which means a variation of up to a certain amount of the number to which reference is being made if the end result is not significantly changed.

Some elements herein may be identified by a part number, which is composed of a base number followed by an alphabetical or subscript-numerical suffix (e.g., 112a, or 112₁). Multiple elements herein may be identified by part numbers that share a base number in common and that differ by their suffixes (e.g., 112₁, 112₂, and 112₃). All elements with a common base number may be referred to collectively or generically using the base number without a suffix (e.g., 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g., a keyboard, a mouse, a touchscreen, and the like), and at least one output device (e.g., a display screen, a printer, a wireless radio, and the like) depending on the nature of the device. For example, and without limitation, the programmable devices (referred to below as computing devices) may be a server, network appliance, embedded device, computer expansion module, a personal computer, laptop, personal data assistant, cellular telephone, smart-phone device, tablet computer, a smart-watch, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional, or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Examples of the system may also be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “user” refers to a user of a user device, and the term “subject” refers to a subject whose measurements are being collected. The user and the subject may be the same person, or they may be different persons in the case where one individual operates the user device, and another individual is the subject. For example, in one embodiment the user may be a health care professional such as a nurse, doctor, rehabilitation specialist, dietitian, researcher, engineer, or other specialist clinician, and the subject is a human.

As referred to herein, real-world data which may be collected outside of a clinical study can include self-reported health metrics and passively collected data. This real-world data may be collected using a software application running on a user device.

Self-reported health metrics may include data from self-reports outside of clinical study, i.e., self-reported health event and symptom input, appointments logged, medications taken, etc.

Passively collected data may be data not received from direct user input which may include: metrics of physiology and behavior (collected passively by remote monitoring with smartwatch, i.e., heart rate, sleep, wheelchair pushes, calories burned, etc.).

Other real-world data can include clinical health outcome data which may be used herein in combination with the software application. This may include, for example, organ health data, organ function data, mental health data, cognitive function data, sleep data, mood data, pain data, sexual function data and mobility data, etc. This clinical health outcome data may be used in order to derive and calibrate predictive aspects of the embodiments herein.

As referred to herein with respect to data there may be input variables, derived variables. These variables may be used as input for modelling, including the machine learning models and goal functions described herein.

Input variables may include self-reported health metrics, passively collected data, and clinical health outcome data as described herein.

Derived variables may include: variables derived from input variables using calculations (area under the curve, binning into categorical/ordinal, one-hot encoding, means, variances, binning, interactions between 2 or more input variables, etc.).

As referred to herein with respect to modelling, there may be machine learning models and goal functions.

Machine learning models may refer to: for example, principal component analysis, factor analysis, logistic regression, decision trees, random forest, support vector machines, neural network, time series analysis, k-nearest neighbors, Bayesian methods, dimensionality reduction, etc. that are used in combination with a similarity metric to calibrate the individualized prediction algorithm using at least one goal function.

Goal functions may refer to a function used as part of the calibration process. The goal function is minimized or maximized for best model performance (e.g., root mean squared error, mean squared error, mean absolute error, adjusted R2 value, Akaike criterion value, cross-entropy error value, p-value based on a test statistic, etc.)

As referred to herein, “mapping” may refer to the identification of the relationships between variables an output. The mapping may be useful for identifying a correlation between variables and a predictive output, or alternatively, for interpreting outputs in view of a set of variable inputs. For example, numerical variable data that is provided may be mapped to distinct categorical or ordinal data.

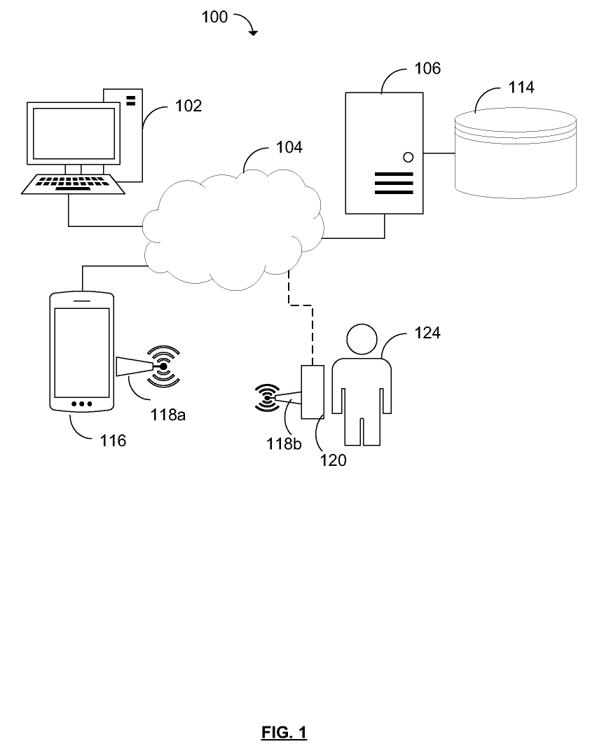

Reference is first made to FIG. 1, which shows a system diagram 100 of a health outcome prediction system for determining a health outcome prediction for a subject 124. The health outcome prediction system includes one or more computer devices 102, a network 104, one or more servers 106, one or more data stores 114, one or more user devices 116 (for example, user devices used by subject 124), and one or more passive data collection monitoring devices 120 proximate to a subject 124.

The one or more computer devices 102 may be used by a user such as a subject, an administrator, clinician, or other medical professional to access a software application (not shown) running on server 106 at remote service over network 104. In one embodiment, the one or more computer devices 102 may access a web application hosted at server 106 using a browser for reviewing health outcome predictions of the subjects 124 using user devices 116. In an alternate embodiment, the one or more user devices 116 may download an application (including downloading from an App Store such as the Apple® App Store or the Google® Play Store) for reviewing health outcome predictions given to the users 124 using user devices 116.

The one or more user devices 116 may be any two-way communication device with capabilities to communicate with other devices. A user device 116 may be a mobile device such as mobile devices running the Google® Android® operating system or Apple® iOS® operating system. A user device 116 may be a smart speaker, such as an Amazon® Alexa® device, or a Google® Home® device. A user device 116 may be a smart watch such as the Apple® Watch, Samsung® Galaxy® watch, a Fitbit® device, or others as known. In some embodiments, the user device 116 and the passive data collection monitoring device 120 are the same.

The one or more user devices 116 may be used by a user 124 to access the software application (not shown) running on server 106 over network 104. In one embodiment, the one or more user devices 116 may access a web application hosted at server 106 using a browser for determining health outcome predictions. In an alternate embodiment, the one or more user devices 116 may download an application (including downloading from an App Store such as the Apple® App Store or the Google® Play Store) for use of the various platform features, including the determination of health outcome predictions. The user device 116 may be a desktop computer, mobile device, or laptop computer. The user device 116 may be in communication with server 106 and may allow a user 124 to review a user profile stored in a database at data store 114, including historical health outcome predictions. The users 124 using user devices 116 may provide user data using a software application and may receive a health outcome prediction based on the passive data collection monitor data and self-reported health metric data as described herein.

The one or more user devices 116 may each have one or more passive data collection monitoring devices. The passive data collection monitors may include a monitoring device 120 in communication with the device 116. The passive data collection monitors may be in wireless communication with the user device 116, for example, using wireless transceiver 118a of the user device 116 and wireless transceiver 118b of the passive data collection monitoring device 120. The user device 116 may include a software application for recording and storing user data in memory. The one or more monitors may include the passive data collection monitoring device 120, an accelerometer, a gyroscope, an electrocardiogram sensor, an electromyogram sensor, an arterial pulse sensor, a temperature sensor, a blood oxygen saturation sensor, a tissue carbon dioxide sensor, an electrical impedance sensor, or a combination thereof. The one or more sensors can also include a camera to capture image data of the user or to capture data of the user’s environment. These sensors may provide sensor data for processing to evaluate passively collected data (physiological and behavioural parameters) related to the subject’s health and medical status.

Accelerometers/gyroscopes are sensors that may be placed on one or multiple areas on the body to measure movement and body orientation, which can evaluate physical activity levels, gait/mobility patterns, posture and balance, as well as paralysis, spasticity, rigidity, tremor, sleep patterns and sleep quality.

Electrocardiograms are sensors that may be placed on at least two regions of the body to measure the electrical activity of the heart, which can evaluate heart rate, heart rate variability, and presence of certain arrythmias.

Electromyographs are sensors that may be placed on one or multiple areas on the body to measure electrical activity of the skeletal muscles. This information is useful for monitoring muscle activity levels and identifying conditions such as paralysis, spasticity, rigidity, and tremor.

Arterial pulse sensors can be in the form of accelerometers, infrared photoplethysmographs, piezoelectric force transducers, and/or doppler ultrasound that can be placed on one or multiple areas on the body to measure pulse arrival times in different segments of the body for evaluation of pulse transit time/pulse wave velocity and blood pressure estimation.

Temperature sensors may be placed on one or multiple areas on the body can measure body temperature, which can be used to evaluate fever or hypothermia.

Blood oxygen saturation sensors may be placed on one or multiple areas on the body to measure the saturation of oxygen in a subject's blood to evaluate the health of the respiratory and cardiovascular system. This information is useful for identifying conditions such as hypoxemia or sleep apnea.

Tissue carbon dioxide sensors can be placed on one or multiple areas on the body to measure transcutaneous carbon dioxide levels to determine the amount of carbon dioxide in a subject's tissue. This information may be useful for evaluating respiratory function and identifying conditions such as hypoventilation, hyperventilation or respiratory failure.

Body electrical impedance sensors may be placed on one or multiple areas on the body can use bioelectrical impedance analysis to determine body composition, including body fluid volumes and fluid shifts. This information may be useful for evaluating hydration status and identifying conditions such as edema or dehydration. Rhythmic impedance changes in the thoracic and/or abdominal regions may also be used to evaluate breathing rate, which is useful to identify conditions of hypoventilation and hyperventilation.

The software application running on the one or more user devices 116 may communicate with server 106 using an Application Programming Interface (API) endpoint and may send and receive passively collected data, self-reported health metric data, user data, mobile device data, and mobile device metadata.

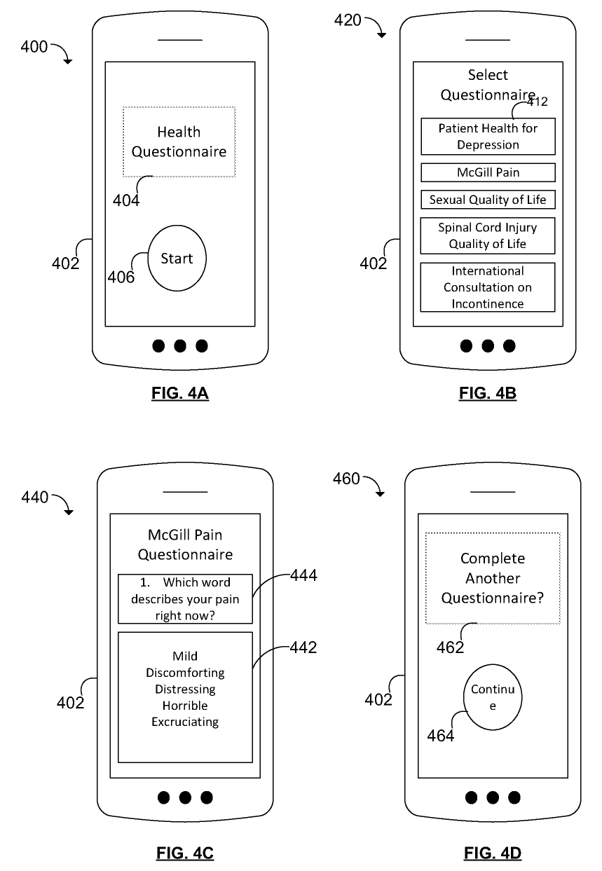

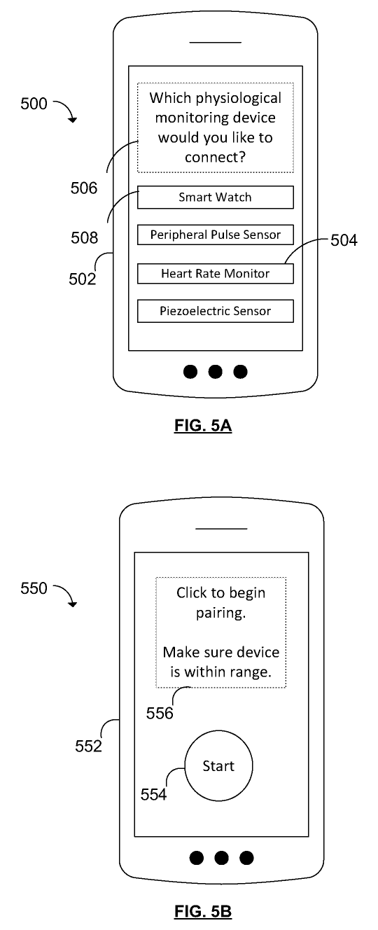

The software application running on the one or more user devices 116 may display one or more user interfaces on a display device of the user device, including, but not limited to, the user interfaces shown in FIGS. 4A, 4B, 4C, 4D, 5A, and 5B.

Local wireless device 118a of the one or more user devices may allow for communication with a local wireless device 118b of one or more monitoring devices including passive data collection monitoring device 120.

The software application running on the one or more user devices 116, or the web application provided by server 106 can assess physiological, behavioral and self-reported markers of health. For example, these markers may include autonomic (i.e., unconscious bodily functions such as blood pressure, bladder function, bowel function, sexual function, etc.) in people without or with disability (such as those using wheelchairs). While such assessment of physiological, behavioral and self-reported markers of health may be provided for people having disability or differing abilities, it may also be provided by non-disabled individuals as well in the form of a health tracking system.

The software application may similarly receive passively collected data. This can include providing a receiving “step count” monitor data or pedometer data, where the “step count” data is converted into a count of “wheelchair pushes”.

The software application running on the one or more user devices 116, or the web application provided by server 106 may also integrate real-world data from clinical health records such that prediction algorithms are generated (e.g., passively collected data/self-report health metric data predicting likelihood of clinical event). Further, other “wearable” monitoring devices may provide passively collected data such as a finger cuff housing a piezoelectric sensor for detecting vascular stiffness (pulse transit time between the phone and the piezoelectric sensor) and other hemodynamic variables may be connected as passive data collection monitoring devices 120. Further, other “implantable” monitoring devices may provide passively collected data such as oxygen sensors, pressure sensors, pH sensors, temperature sensors, electrochemical biosensors, cardiac rhythm management devices, neural sensors, telemetry capsules, lactate sensors, coagulation sensors, respiration rate sensors, humidity sensors, flow sensors, and biomarker sensors.

The software application running on the one or more user devices 116, or the web application provided by server 106 may provide for telecommunication or telehealth opportunities. For example, connectivity to clinician users using video conferencing, audio conferencing, etc. may be used to provide a direct line of communication between subjects and clinical personnel, or the subjects’ medical providers.

The software application running on the one or more user devices 116, or the web application provided by server 106 may also provide a portal with educational resource for users.

The passive data collection monitoring device 120 may be a may be a wearable device, a smartphone, an accelerometer, a gyroscope, an electrocardiogram sensor, an electromyogram sensor, an arterial pulse sensor, a temperature sensor, a blood oxygen saturation sensor, a tissue carbon dioxide sensor, an electrical impedance sensor, a peripheral pulse sensor, or a smart watch such as the Apple® Watch, Samsung® Galaxy® watch, a Fitbit® device, a camera (including a sensor generating image and video signals) or others as known. The passive data collection monitoring device 120 may transmit passively collected data proximate to the user 124 to the user device 116 and may receive alarms or notifications from the user device 116 for presentation to the user 124. The passive data collection monitoring device 120 may be worn on the body of user 124, on their clothing, or may be positioned proximate to the user 124. In some embodiments, the user device 116 and the passive data collection monitoring device 120 are the same.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 116 and the one or more computer devices 102. The server 106 may further be in communication with a database at data store 114. The database at data store 114 and the server 106 may be provided on the same server device, may be configured as virtual machines, or may be configured as containers. The server 106 and a database at data store 114 may run on a cloud provider such as Amazon® Web Services (AWS®), Microsoft® Azure, or Google® Cloud Platform.

The server 106 may host a web application or an Application Programming Interface (API) endpoint that the one or more user devices 116 may interact with via network 104. The server 106 may make calls to the user device 116 or the passive data collection monitoring device 120 to poll for passive data collection monitoring data. In an alternate embodiment, the user device 116 or the passive data collection and monitoring device 120 may push data to the server 106 for processing. The server 106 may make calls to the database at data store 114 to query subject data, passively collected data, self-reported health metrics, health outcome model data, or other data received from the users 124 of the one or more user devices 116. The requests made to the API endpoint of server 106 may be made in a variety of different formats, such as JavaScript Object Notation (JSON), Graph Query Language (GraphQL) or eXtensible Markup Language (XML). The API endpoint of server 106 may be a Representational State Transfer (REST) API.

The database of data store 114 may store subject information including passively collected data, self-reported health metrics, subject and/or user information including subject and/or user profile information, configuration information, and clinical health outcome data. The database of data store 114 may be a Structured Query Language (SQL) such as PostgreSQL or MySQL or a not only SQL (NoSQL) database such as MongoDB.

The passive data collection monitoring device 120 may be connected to network 104 directly and may send and receive data from the server 106. In an alternate embodiment, the passive data collection monitoring device 120 may send and receive data with the user device 116 using a local wireless network 118a and 118b such as Bluetooth.

The user device 116 may further prompt the user 124 to provide data using one or more questions in a self-report assessment form (see e.g., FIGS. 4 and 5). The self-reported health metrics may be collected at regular intervals, or according to a predetermined schedule.

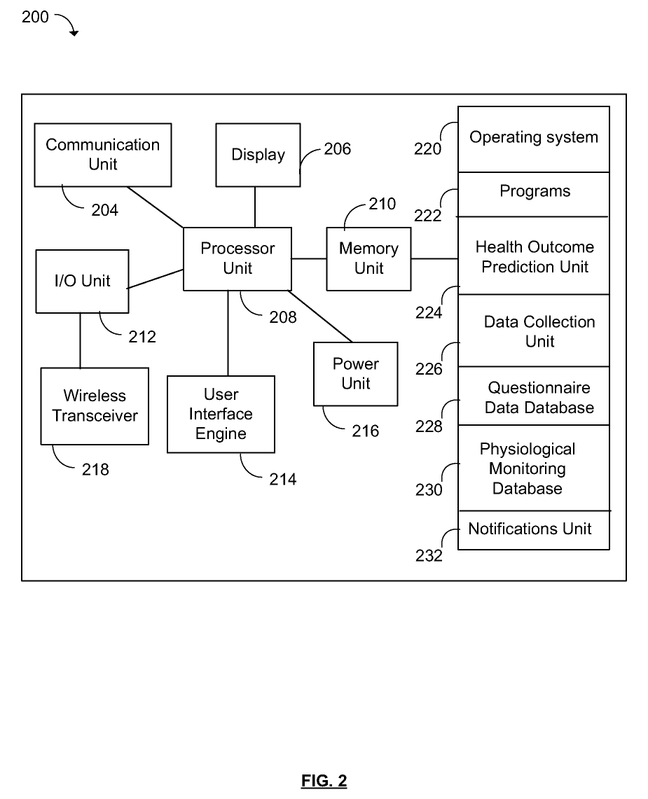

FIG. 2 shows a user device diagram 200 showing detail of the one or more user devices in 116 in FIG. 1.

The user device 200 includes one or more of a communication unit 204, a display 206, a processor unit 208, a memory unit 210, I/O unit 212, a user interface engine 214, a power unit 216, and a wireless transceiver 218. The user device 200 may be a laptop, gaming system, a mobile device such as an Android® or iOS® device, smart speaker device, mobile phone device, smart watch or others as are known.

The communication unit 204 can include wired or wireless connection capabilities. The communication unit 204 can include a radio that communicates utilizing CDMA, GSM, GPRS or Bluetooth protocol according to standards such as IEEE 802.11a, 802.11b, 802.11g, or 802.11n. The communication unit 204 can be used by the user device 200 to communicate with other devices or computers.

The communication unit 204 may provide communications over the local wireless network using a protocol such as Bluetooth (BT) or Bluetooth Low Energy (BLE). Communication unit 204 may communicate with the wireless transceiver 218 to transmit and receive information via local wireless network with the passive data collection monitoring device (see e.g., device 120 in FIG. 1).

The display 206 may be an LED or LCD based display and may be a touch sensitive user input device that supports gestures.

In an alternate embodiment, the user device 200 may be a voice interaction system such as Alexa®, Google® Home®, or Apple® Siri® based device. In this case, the display may be implemented using a speaker, or a combination of a speaker and a display. The input to the user device in this embodiment may be a touch screen, a microphone, or a combination of a touch screen and a microphone.

The processor unit 208 controls the operation of the user device 200. The processor unit 208 can be any suitable processor, controller or digital signal processor that can provide sufficient processing power depending on the configuration, purposes, and requirements of the user device 200 as is known by those skilled in the art. For example, the processor unit 208 may be a high-performance general processor. In alternative embodiments, the processor unit 208 can include more than one processor with each processor being configured to perform different dedicated tasks. In alternative embodiments, it may be possible to use specialized hardware to provide some of the functions provided by the processor unit 208. For example, the processor unit 208 may include a standard processor, such as an Intel® processor, an ARM® processor or a microcontroller.

The processor unit 208 can also execute a user interface (UI) engine 214 that is used to generate various UIs, some examples of which are shown and described herein, such as interfaces shown in FIGS. 4A-4D and 5A-5B.

The present systems, devices and methods may provide an improvement in the operation of the processor unit 208 by ensuring the analysis of passively collected data is performed using collected variables. The reduced processing required for the collected variables in the analysis (as compared with processing the superset of all collected variables) reduces the processing burden required to make health outcome predictions based on a individualized prediction algorithm.

The memory unit 210 comprises software code for implementing an operating system 220, programs 222, health outcome prediction unit 224, data collection unit 226, self-reported health metric database 228, passively collected data database 230 and notifications unit 232.

The memory unit 210 can include RAM, ROM, one or more hard drives, one or more flash drives or some other suitable data storage elements such as disk drives, etc. The memory unit 210 is used to store an operating system 220 and programs 222 as is commonly known by those skilled in the art.

The I/O unit 212 can include at least one of a mouse, a keyboard, a touch screen, a thumbwheel, a trackpad, a track-ball, a card-reader, an audio source, a microphone, a monitoring device, voice recognition software and the like again depending on the particular implementation of the user device 200. In some cases, some of these components can be integrated with one another.

The user interface engine 214 is configured to generate interfaces for users to configure passively collected data, complete self-reported health metrics , connect to the passive data collection monitoring device, view physiological, behavioral, and self-reported health metric data, view individualized health outcome predictions, etc. The various interfaces generated by the user interface engine 214 are displayed to the user on display 206.

The power unit 216 can be any suitable power source that provides power to the user device 200 such as a power adaptor or a rechargeable battery pack depending on the implementation of the user device 200 as is known by those skilled in the art.

The operating system 220 may provide various basic operational processes for the user device 200. For example, the operating system 220 may be a mobile operating system such as Google® Android® operating system, or Apple® iOS® operating system, or another operating system.

The programs 222 include various user programs so that a user can interact with the user device 200 to perform various functions such as, but not limited to, viewing passively collected data measurement and self-reported health metrics, receiving, and viewing passively collected data from the monitoring device, receiving any data related to individualized health outcome predictions, as well as receiving messages, notifications, and alarms as the case may be.

The programs 222 may include a telephone calling application, a voice conferencing application, social media applications, and other applications as known. The programs 222 may make calls, requests, or queries to the prediction unit 224, the data collection unit 226, the self-reported health metric database 228, the passively collected outcome database 230, and the notifications unit 232. The programs 222 may be downloaded from an application store (“app store”) such as the Apple® App Store® or the Google® Play Store®.

In one or more embodiments, the programs 222 may include a passive health outcome application that may transmit passively collected data to a server, optionally receive health outcome predictions, and optionally provide alerts to the user’s device to the user automatically and without user prompting. In one or more embodiments, the passive monitoring device application may be connected wirelessly to a user device such as a mobile phone, and may cause an email, text message, or application notification to be displayed to a user on the user device.

In one or more embodiments, the programs 222 may include a subject tracker for a plurality of subjects. The subject tracker may provide a user interface providing information and health outcome predictions from the subjects. The health outcome predictions may be provided to the medical professional in order to for example, collect health data or adjust a treatment plan for a subject in the plurality of subjects. The user interface may include a reporting interface for the plurality of subjects, or alternatively may provide email, text message, or application notifications to the medical professional about one or more subjects based on subject health outcome predictions.

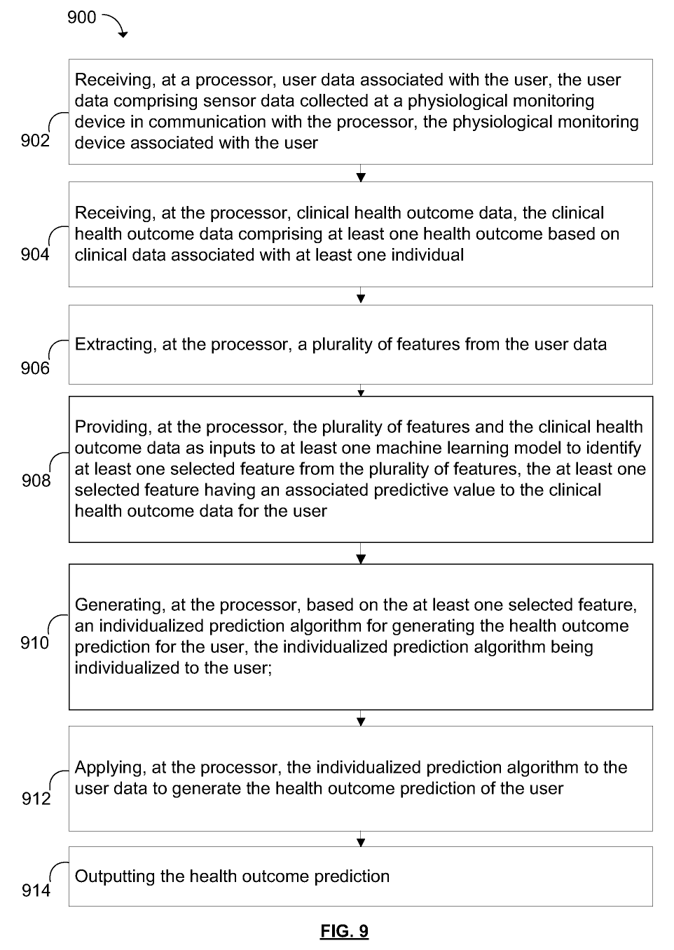

The health outcome prediction unit 224 receives passively collected monitoring data from the monitoring device connected to I/O unit 212 via the data collection unit 226 and may transmit the passively collected monitoring data to the server (see e.g., 106 in FIG. 1). In response, the server may operate the method as described in FIG. 9 to generate a health outcome prediction for the subject and may respond with the health outcome prediction to the user device. The passively collected data may be stored in the real-world database 230 along with the prediction data.

The health outcome prediction unit 224 may use self-reported health metric data in the real-world database 228, passively collected data in the passively collected database 230, or may receive predictions made at server 106 (see FIG. 1). The health outcome prediction unit 224 may predict one or more health outcome predictions for a subject and these may be provided as notifications to the user of device 200 using notification unit 232. The health outcome prediction unit 224 may determine an individualized prediction algorithm to be used for a subject based on the data in databases 228 and 230.

The health outcome prediction unit 224 receives clinical health outcome data including at least one health outcome based on clinical data associated with the subject or user of the user device 200. For example, the health outcome prediction unit 224 may receive the clinical health outcome data from the server 106 (see FIG. 1).

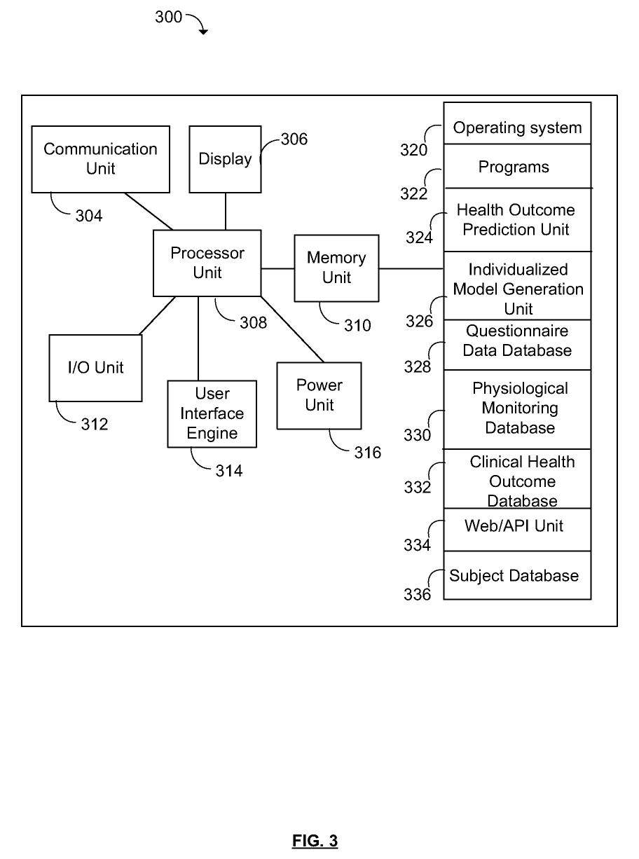

The clinical health outcome data generally corresponds to the data stored in the clinical health database 332 (see FIG. 3).

The data collection unit 226 receives passively collected data from the monitoring device connected to the I/O unit 212. The data collection unit 226 may pre-process received data from a monitoring device such as the passive data collection monitoring device (see e.g., 120 in FIG. 1), this may include averaging, smoothing, or removing noise from the passively collected data.

In one or more embodiments, the data collection unit 226 receives passively collected data from the monitoring device via the wireless transceiver 218. The data collection unit 226 may receive the passively collected data and may store it in the passively collected data database 230. The data collection unit 226 may receive the passively collected data and may transmit it to a server. The data collection unit 226 may supplement the passively collected data that is received from the monitoring device with mobile device data and mobile device metadata. The data collection unit 226 may further send passively collected data to the server. The data collection engine 226 may communicate with the monitoring device wirelessly, using a wired connection, or using a computer readable media such as a flash drive or removable storage device.

The self-reported health metric database 228 may be a Structured Query Language (SQL) such as PostgreSQL or MySQL or a not only SQL (NoSQL) database such as MongoDB.

The self-reported health metric database 228 may be a database for storing self-reported health metric responses received by the user device 200. The self-reported health metric database 228 may receive the data from the data collection unit 226. The self-reported health metric data may be prompted to a user of the user device 200 using a user interface such as the ones in FIGS. 4 and 5.

The self-reported health metric database 228 may include self-reported health metric data obtained from the health outcome questionnaires (see e.g., FIGS. 4 and 5). These data may include information on subject health events, symptoms, medication use, and overall quality of life. Example data to be obtained from these data can include information on symptoms/or other self-reported feedback related to low and high blood pressure, bladder function, bowel function, sexual function, sleep quality, fatigue, pressure sores, body temperature, pain, bone health, spasticity, and sweating and/or goosebumps, and can as well ask questions related to a subject’s daily performance, using questions related to their concentration and thinking ability, productivity, mood, and social engagement. This self-reported health metric data may be collected through a subject-oriented online-based platform with subject access granted in the form of a mobile phone application and/or desktop computer.

The passively collected data database 230 may be a Structured Query Language (SQL) such as PostgreSQL or MySQL or a not only SQL (NoSQL) database such as MongoDB.

The passively collected data database 230 may be a database for storing passively collected data received from monitoring devices, whether monitoring devices are integrated with the device 200 or whether the monitoring devices are in communication with the device 200 using communication unit 204, either via a network connection or via a local wireless network connected using wireless transceiver 218.

The notification unit 232 may provide notifications to the user or subject using user device 200. These notifications may be delivered by the operating system 220 to the user. Additionally, the notifications may provide information based on the health outcome prediction created by the health outcome prediction unit as described in FIG. 9.

FIG. 3 shows a server diagram showing detail of the server 106 in FIG. 1. The server 300 includes one or more of a communication unit 304, a display 306, a processor unit 308, a memory unit 310, I/O unit 312, a user interface engine 314, and a power unit 316.

The communication unit 304 can include wired or wireless connection capabilities. The communication unit 304 can include a radio that communicates using standards such as IEEE 802.11a, 802.11b, 802.11g, or 802.11n. The communication unit 304 can be used by the server 300 to communicate with other devices or computers.

Communication unit 304 may communicate with a network, such as networks 104 from FIG. 1.

The display 306 may be an LED or LCD based display and may be a touch sensitive user input device that supports gestures.

The processor unit 308 controls the operation of the server 300. The processor unit 308 can be any suitable processor, controller or digital signal processor that can provide sufficient processing power depending on the configuration, purposes, and requirements of the server 300 as is known by those skilled in the art. For example, the processor unit 308 may be a high-performance general processor. In alternative embodiments, the processor unit 308 can include more than one processor with each processor being configured to perform different dedicated tasks. The processor unit 308 may include a standard processor, such as an Intel® processor or an AMD® processor.

The processor unit 308 can also execute a user interface (UI) engine 314 that is used to generate various UIs for delivery via a web application provided by the Web/API Unit 334, some examples of which are shown and described herein, such as interfaces shown in FIGS. 4A-D.

The memory unit 310 comprises software code for implementing an operating system 320, programs 322, health outcome prediction unit 324, individualized model generation unit 326, passively collected data database 328, self-reported health metric database 330, clinical health outcome database 332, Web/API unit 334, and subject database 336.

The memory unit 310 can include RAM, ROM, one or more hard drives, one or more flash drives or some other suitable data storage elements such as disk drives, etc. The memory unit 310 is used to store an operating system 320 and programs 322 as is commonly known by those skilled in the art.

The I/O unit 312 can include at least one of a mouse, a keyboard, a touch screen, a thumbwheel, a trackpad, a track-ball, a card-reader, an audio source, a microphone, voice recognition software, a brain-computer interface, an artificial intelligence prompt based on user-behavior, and the like again depending on the particular implementation of the server 300. In some cases, some of these components can be integrated with one another.

The user interface engine 314 is configured to generate interfaces for users to configure the passively collected monitoring device, record self-reported health metrics, view passively collected monitoring data, view self-reported data, view health outcome predictions, etc. The various interfaces generated by the user interface engine 314 may be transmitted to a user device by virtue of the Web/API Unit 334 and the communication unit 304.

The power unit 316 can be any suitable power source that provides power to the server 300 such as a power adaptor or a rechargeable battery pack depending on the implementation of the server 300 as is known by those skilled in the art.

The operating system 320 may provide various basic operational processes for the server 300. For example, the operating system 320 may be a server operating system such as Ubuntu® Linux, Microsoft® Windows Server® operating system, or another operating system.

The programs 322 include various user programs. The programs 322 may provide a public health platform that is web-based, or client-server-based application via Web/API unit 334 that provides for health research on a large population of subjects. The public health platform may provide access for queries and data analysis of the passively collected data database 328, the self-reported health metrics database 330, and the subject database 336. The health platform may allow for population health research on different groups, including based on demographic information, and the subject’s health functions.

The health outcome prediction unit 324 generally corresponds to the health outcome prediction unit 224 (see FIG. 2) and the prediction using the method of FIG. 9 may occur at either the user device 200 or the server 300.

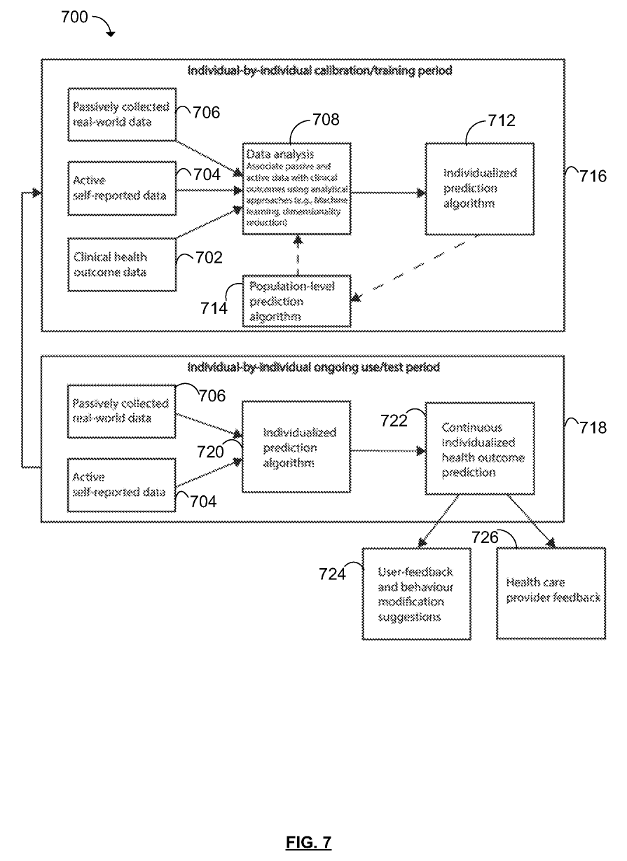

The individualized model generation unit 326 may operate using the method of FIG. 7, along with the databases 328, 330, 332 and 336 in order to generate individualized models for the subjects using the platform. The generated individualized models may be used by an individualized algorithm and health outcome predictions may be created based on the method in FIG. 9. The generated individualized models may be stored in subject database 336, and may also be transmitted to the user devices (see e.g., devices 116 in FIGS. 1 and 200 in 2).

The self-reported health metric database 328 generally corresponds to the self-reported health metric database 228 (see FIG. 2). The self-reported health metric database 328 however may include self-reported health metric data for a plurality of subjects using a plurality of user devices that are received via communication unit 304.

The passively collected data database 330 generally corresponds to the passively collected data database 230 (see FIG. 2). The passively collected data database 330 however may include passively collected data for a plurality of subjects using a plurality of user devices that are received via communication unit 304.

The clinical health outcome database 332 contains clinical health outcome data, the clinical health outcome data includes at least one health outcome based on clinical data. The clinical data may be associated with a plurality of subjects, for example, the plurality of users of the plurality of user devices.

The clinical health outcome data in database 332 may include data generated from a subject’s medical history. For example, the clinical health outcome data may include organ health data, organ function data, mental health data, cognitive function data, sleep data, mood data, pain data, sexual function data and mobility data, etc. This may be received from an electronic medical records (EMR) system.

The clinical health outcome data in database 332 may include, but is not limited to: Hospital/Medical Center Admissions (including emergency room, in-patient, outpatient facilities, and long-term care) for health events. Surgeries indicated (i.e., coronary artery bypass graft(s), indwelling urinary catheter placements, etc.). Interventions prescribed (i.e., ablations, catheterization, Botox injection, wound dressings, lung treatment, thrombectomy, etc.). Diagnoses (i.e., arteriosclerosis, mood disorders, cancer, myocardial infarction, diabetes, etc.) Diagnostic imaging results (i.e., cardiac MRI, MRI, CT scan, DEXA, x-ray, ultrasound, echocardiography, etc.). Diagnostic testing results (i.e., nerve conduction study, electrocardiogram, urodynamics, stress test, phlebotomy, fecal analysis, urinalysis, psychoanalysis, biopsies, etc.). Other outcomes from clinical testing (i.e., neurological scores, heart health measures, results from blood, urine, cerebral spinal fluid, outcomes from imaging (i.e., DEXA values, cardiac MRI measures, echocardiography, ultrasonography, etc.)).

Medication use (prescribed, dispensed, renewed, changed, terminated). Specialist care accessed (Cardiology, Urology, Gastroenterology, Physiatry, Dietetics, Psychiatry, Sleep specialist, Pain specialist, Sexual Health, Obstetrics and Gynecology, Proctology, Neurology, Neurosurgery, General Surgery, Dermatology, etc.)

The clinical health outcome data in database 332 may be obtained through the subject’s medical/health history in correspondence with the subject’s health care provider(s).

The clinical health outcome data in database 332 may include standard EMR records, free text, test result data, clinician generated information, and other information. The clinical health outcome database 332 may be a Structured Query Language (SQL) such as PostgreSQL or MySQL or a not only SQL (NoSQL) database such as MongoDB.

The Web/API unit 334 may be a software application that provides the platform functionality over a network, such as via the communication unit 304. The Web/API unit 334 may provide a web application interface to users who access the server 300 through a network. The Web/API unit 334 may provide an Application Programming Interface (API) to software applications that are running on user devices (such as devices 116 in FIG. 1 and 200 in FIG. 2).

The subject database 336 may include user account information, including subject registration information, information relating to the user devices that each user uses and the associated monitoring devices. The subject database 336 may further include generated individualized models for the associated subjects involved in data collection generated by the individualized model generation unit 326. The subject database 336 may also include records associated with captured metrics relating to user interactions with the user interface engine 314 from figures, e.g., time spent on specific questionnaires, specific resources opened.

Referring next to FIGS. 4A, 4B, 4C, and 4D together, several user interface diagrams are shown in accordance with one or more embodiments.

As described herein, users of user devices (e.g., devices 116 in FIGS. 1 and 200 in FIG.2) may have data collected by a monitoring device and may voluntarily provide self-reported health metric information contemporaneously to the passively collected data collection. The self-reported health metrics may be initiated by way of a notification at a predetermined interval or schedule to the user of the user device. The type of questions provided in the self-report may be selected by a domain-knowledge expert, a clinician monitoring the subject or may be selected by a clinical study designer.

In response to a notification to complete a self-report on health metrics, the user of the user device may open a web application hosted by the server in a browser or may open a software application running on the user device in order to answer the self-report prompts.

These user interfaces improve the conventional systems for collecting self-reported health metric data, and the self-reported health metric data herein is collected contemporaneously with passively collected data. The user-interfaces provide several advantages, such as improved accessibility features for individuals living with disabilities that can include wheelchair users, mobility impairments, neurological diseases, as well as individuals without disability. The user-interface advantages also include simplified delivery of abbreviated questions to users, push notifications to increase compliance, app utilization metrics, passive data collection using monitoring devices including wearable and implantable monitoring technology, as well as easily accessible educational resources. This is complimented by the ability to provide individualized health outcome predictions to the user using their user device.

Referring to FIG. 4A, there is shown a user interface 400 on a user device 402 including a self-reported health metric landing page. In response to the notification of the user to complete a self-reported health metric, the user may be provided with self-reported health metric information 404 and a start button 406.

Referring to FIG. 4B, there is shown a user interface 420 of a self-reported health metric listing page. Once the start button 406 in FIG. 4A is selected the user at user device 402 may be provided with one or more self-reported health metric options, for example, a mental health questionnaire option 412. The user may select a self-reported health metric option 412 from the list.

Referring to FIG. 4C, there is shown a user interface 440 of a self-reported health metric prompt page. The subject or user using the user device 402 may self-report answers 442 to the self-reported health metric prompts 444. There may be a series or sequence of question prompts 444 that are provided for a user to provide answers 442 to.

Referring to FIG. 4D, there is shown a user interface 460 of a self-reported health metric completion page. Once the self-reported health metric has had questions answered, a completion message 462 may be presented along with a continue button 464 which, when clicked or selected, returns the user to the self-reported health metric options interface in FIG. 4B. A user may receive notifications to complete many different self-reported health metric and the interfaces herein may enable them to efficiently self-report self-reported health metric data in conjunction with the collection of the passively collected data.

Referring next to FIGS. 5A and 5B together, there is shown an interface 500 for connecting a monitoring device such the passive data collection monitoring device (see e.g., 120 in FIG. 1) and a pairing interface 550 in accordance with one or more embodiments.

The interface 500 may provide instructions 506 and different connection options 504 for the user to select at the user device 502 in order to receive monitor data. For example, a heart rate monitor 504 or smart watch 508 may be selected and connected. The monitoring devices used for connection may be those described for the monitoring device 120 in FIG. 1.

The interface 550 may include particular pairing instructions 556 for the selected monitoring device, as well as a pairing start button 554 used to initiate the pairing of the devices. This may include the wireless communication based on wireless transceivers 118 (see FIG. 1).

The interface 500 may also provide for integration health applications running on the user device such as Apple Health, Google Health Connect, or 3rd Party Applications that collect health data from a passive data collection monitoring device e.g., Strava, Garmin Connect, MyFitnessPal. The interface in this case may enable a user to connect or link user accounts with the 3^{rd} party application.

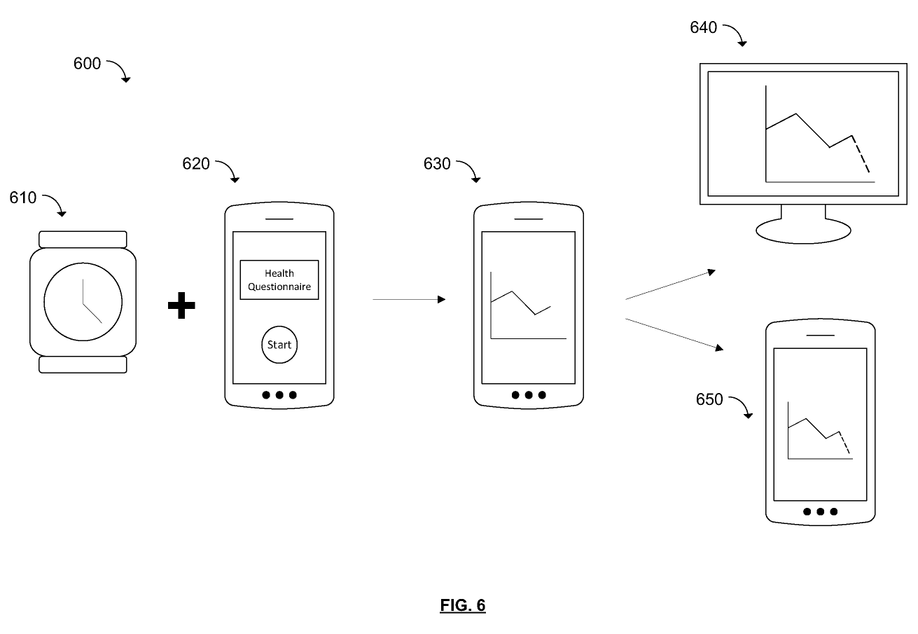

FIG. 6 shows an example user interaction workflow diagram 600 in accordance with one or more embodiments.

A subject of a study may participate in data collection and may receive outcome predictions. This may include the subject having one or more monitoring devices 610 proximate to, or attached to, their body in order to passively collect data about the subject. Contemporaneously, the subject may receive notifications on their user device 620 with requests to provide answers to self-reported health metrics. The subject may participate in the study over a period of time, for example, a day, a week, a month, or several months. The passively collected data and the self-reported health metric data may be received and stored in a database and may store subject metrics that are collected over time. These metrics may be tracked by a clinician, data analyst, the subject, or another user over time using a reporting interface 630.

The metric reporting 630 may include a clinician or study administrator interface 640 that may be available to subjects. The metric reporting interface 640 may further include outcome predictions that have been made to the subject.

The metric reporting 630 may also include a subject or user interface 650 that provides for metric reporting to the subject. The subject reporting interface 650 may also include the outcome predictions that have been made to the subject during the course of the study.

FIG. 7 shows a flowchart of an individualized prediction method in accordance with one or more embodiments. The method of FIG. 7 may be executed at a user device of a subject, at a server (for example, server 106 see FIG. 1), or a combination thereof. The individual and personalized analysis portion 716 of the method 700 may be performed at a server (e.g., server 106 in FIG. 1) and the inference/prediction portion 718 may be performed at a user device (e.g., user device 116 in FIG. 1).

The individual and personalized analysis portion 716 may be for creating models to operate individualized prediction algorithms for continuous health outcome predictions using passively collected data and self-reported health metric data. The individual and personalized analysis portion 716 may be a calibration/testing period where passively collected data 706 and self-reported health metric data 704 is obtained from subjects.

For each subject, an individual and personalized analysis portion 716 is conducted in order to calibrate and train an individualized model 712 for making inferences or predictions about that subject. The generated individualized model 712 from individual and personalized analysis portion 716 may be transmitted to a user device and used in inference/prediction portion 718. The health outcome prediction 722 may further be provided as user feedback 724 on a user device or provided to a health care provider interface 726.

At 702, clinical health outcome data is received. The clinical health outcome data 702 may be the clinical health outcome data in database 332 (see FIG. 3), e.g., as direct inputs, or by considering derived calculations, such as means, areas-under-the-curve, standard deviations, etc.

At 704, self-reported health metric data may be obtained(as described in FIGS. 4A – 4D and self-reported health metric databases 228 and 328) in accordance with an embodiment of the present disclosure. This can include self-reported health metric data that can include information on subject health events, symptoms, medication use, and overall quality of life. Example data to be obtained from these self-reported health metrics can include information on events, symptoms/or other self-reported feedback related to low and high blood pressure, bladder function, bowel function, sexual function, sleep quality, fatigue, pressure sores, body temperature, pain, bone health, spasticity, and sweating and/or goosebumps. The self-reported health metrics may further include questions related to a subject’s daily performance, using questions related to their concentration and thinking ability, productivity, mood, self-care, and social engagement. These self-reported health metrics may be administered through a subject-oriented online-based platform such as the web application provided by server 106 (see FIG. 1) or by a subject accessing a mobile application or desktop application on their user device.

At 706, passively collected data are received from a monitoring device (e.g., received from monitoring device 120 in FIG. 1, and stored in the passively collected data database 230 and 330). For example, the passive data collection monitoring device may be a physiological or behavioral monitoring device. Passively collected data may be obtained from the monitoring device as described herein with respect to the monitoring device 120 in FIG. 1. The monitoring device may be configured to communicate with a companion user device via a wired or wireless communication channel (e.g., Bluetooth, Wi-Fi, etc. as described at 218 in FIG. 2) in order to output information. In some embodiments, the display module of the user device may be a smartphone, tablet computer, or similar portable computing device made accessible to the user throughout typical day-to-day activities.

At 708, machine learning analysis of the clinical health outcome data 702, the self-reported health metric data 704, and the passively collected data 706 may occur. This may include associating the various self-reported health metric 704 and passively collected data 706 with clinical health outcome data 702. The machine learning analysis may include using different analytical approaches, which may include one or more machine learning approaches: logistic regression, decision trees, random forest, support vector machine, neural network, time series analysis, k-Nearest neighbors, Bayesian method, and/or other machine learning/ dimensionality reduction approaches. This analysis then outputs an individualized health outcome prediction model 712 that may be used by an individualized prediction algorithm.

From each of these data sources (702, 704 and 706), both raw data and derived calculations may be used to generate derived variables for the machine learning model.

The derived variables are derived calculations using combinations of variables that may include passively collected data variables such as physical activity level (# of minutes engaged in exercise, # of steps, #of calories burned/day, # of time standing, basal energy burned, distance covered, breathing rate, transcutaneous carbon dioxide level), body composition (self-reported height and weight, body mass index, body fat percentage, resting metabolic rate, total water content), gait pattern (asymmetry, cadence, # of steps, fall detection), sleep pattern (# of minutes total sleep, # minutes disrupted sleep, # of minutes in a deep sleep, # of minutes restful), sleep quality (total restful sleep time, average sleeping heart rate, % sleep disruption, average sleeping heart rate variability, temperature, sweating, average sleeping blood oxygenation, sleep quality index, breathing rate, sleep-related breathing problems, sleep efficiency), cardiovascular health (resting heart rate, rate during exercise, heart rate variability, presence of arrhythmia, presence of bradycardia or tachycardia, blood oxygenation, pulse transit time, pulse wave velocity, electrocardiogram), blood pressure (systolic blood pressure, diastolic blood pressure, mean arterial pressure, variability in pressure). The derived variables are derived calculations using combinations of variables that may also include self-reported health metrics such as bladder health (bladder function, urination rate, number of catheterizations), bowel health (bowel health, bowel routine time), sexual function, pain, bone health, cognitive function, mood and concentration, mental health, mindfulness metrics), well-being, the incidence and severity of headache, skin health (self-reported, images) body temperature (incidence and severity), sweating, nutrition, mobility, self-care, productivity, and app interactions (reaction time, reading time, changes to resources accessed, etc.).

The derived calculations may further include minutes of exercise, blood glucose, electrodermal activity, waist circumference, distance walking or running, flights of stairs climbed, moving minutes, distance travelled, mindfulness, consumed water volume, audiogram, etc.

The derived calculations may further include averaging, smoothing, binning, mapping, encoding, categorizing, filtering or transforming of the raw data to generate new variables for the data analysis 708.

The derived variables may be referred to herein as features, for example as described in FIG. 9.

In at least one embodiment, data may be normalized to have zero-mean and unit variance and a principal component analysis is performed.

In at least one embodiment, the 3 most similar loadings to the desired clinical outcome may be identified using cosine similarity in the dimensionally reduced space. A generalized linear model may be used to fit the 3 most similar loadings to the clinical outcome. Next, an iterative approach may be used to improve goodness-of-fit, by considering additional loadings in the dimensionality reduced space until a final model 712 is found that has the optimized values on the goal functions, such as root-mean square error and Akaike Information Criterion. Other models may be used, such as neural network-based models and regression models.

The individualized prediction algorithm 720 may provide health outcome predictions 722 from the passively collected monitoring data 706 alone, or alternatively from the self-reported health metric data 704, or a combination of both passive data 706 and self-reported data 704 without needing clinical health outcome data 702.

The health outcome prediction 722 may provide users with real time health monitoring feedback, comprised of potential changes in risk, as well as suggested behavior change/need for intervention with a health-care provider. The individualized health outcome prediction 722 may be outputted to a display 206 (see e.g., FIG. 2) as user feedback, suggestion to make appointment with health care provider, and behavior modification suggestions 724. The health outcome predictions 722 may be provided as a singular assessment score or in categories associated with each component of the individual’s health. The display 206 or other communication medium may communicate to the user in the form of an application on the user device, or alternatively as an online-based platform (such as the one provided by server 106, see FIG. 1) with subject access by a user device 116 (see FIG. 1).

The generated individualized or personalized model 712 may be transmitted to the inference prediction portion 718 on the user device in order to enable inferences or predictions about the subject’s clinical outcomes.

The inference prediction portion 718 includes the passively collected data 706 and the self-reported health metric data 704, the individualized prediction algorithm 720 which uses the individualized or personalized model generated at 712 using the passively collected data 706 and the self-reported health metric data 704 to generate a health outcome prediction 722. The health outcome prediction 722 may include a notification to the subject at the user device.

The health outcome prediction 722 may be presented to a subject using a user device, such as user device 116 (see FIG. 1). The display 206 (see FIG. 2) of the user device 116 may receive input from the user to customize the type of information displayed. The user input can be in the form of touch screen operation, standard computer commands, and/or in other ways, such as by receiving voice commands via a microphone.

This individualized prediction algorithm 720 may use the model created in the training portion 716 in combination with an algorithm to provide the clinical predictions for the subject, which may be transmitted to the server 106 (see FIG. 1) and may be incorporated into population-level data sets 714 to refine the individual prediction algorithm in the future (e.g. combined with other data in order to improve model accuracy).

The method 700 may provide for the ongoing collection of passively collected data 706 and self-reported health metric data 704 may be transmitted to the server 106 to improve individualized prediction algorithms when they are trained. Iterative looping logic between portions 716 and 718 may update and “validate” the individualized models developed in portion 716.

At 722, the clinical prediction may be delivered at an output device to the user device, for example, user device 116 (see FIG. 1).

In addition, the recommendation shown to the subject on display 206 can provide feedback to the user, which could be in the form of behavioral change recommendations, such as physical activity recommendations, sleep practices based on past collected data, notifying individuals of how to manage high or low blood pressure days, and when it may be necessary to follow up with a health care practitioner regarding a particular health risk that has changed over time.

The display 206 can also provide users with information about the signal quality received from connected wearable monitoring devices 120 (see FIG. 1) used for the collection of passively collected data, and based on the issues detected, can inform the user on an approach to improve signal quality coming from one or more of the connected sensors within the wearable monitoring devices.

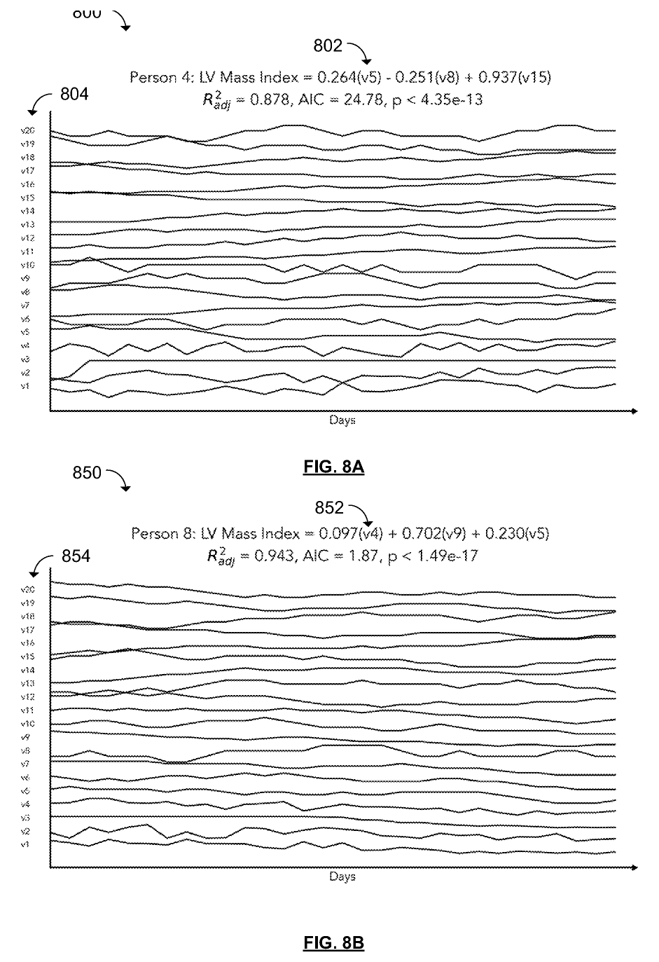

Referring next to FIG. 8A, there is shown an example diagram 800 of a individualized prediction algorithm 802 with daily tracking of different variables 804 in accordance with one or more embodiments.

Referring next to FIG. 8B, there is shown another example diagram 850 of a individualized prediction algorithm 852 with daily tracking of different variables 854 in accordance with one or more embodiments.

In FIGS. 8A and 8B, the variables 804 and 854 respectively including the following: Mean heart rate: v1, Daily step count: v2,

O2 saturation: v3, Heart rate variability: v4, Area Under the Curve (AUC) for Low Blood-Pressure: v5, which may be determined by taking the definite integral of data for a given variable, AUC for High Blood-Pressure: v6, AUC for Bladder Function: v7, AUC for Bowel Function: v8, AUC for Sexual Function: v9, AUC for Sleep Quality: v10, AUC for Fatigue: v11, AUC for Pressure Sores: v12, AUC for Body Temperature: v13, AUC for Sweating: v14, AUC for Concentration: v15, AUC for Mood: v16, AUC for Pain: v17, AUC for Bone Health: v18, AUC for Spasticity: v19, AUC for Well Being: v20.

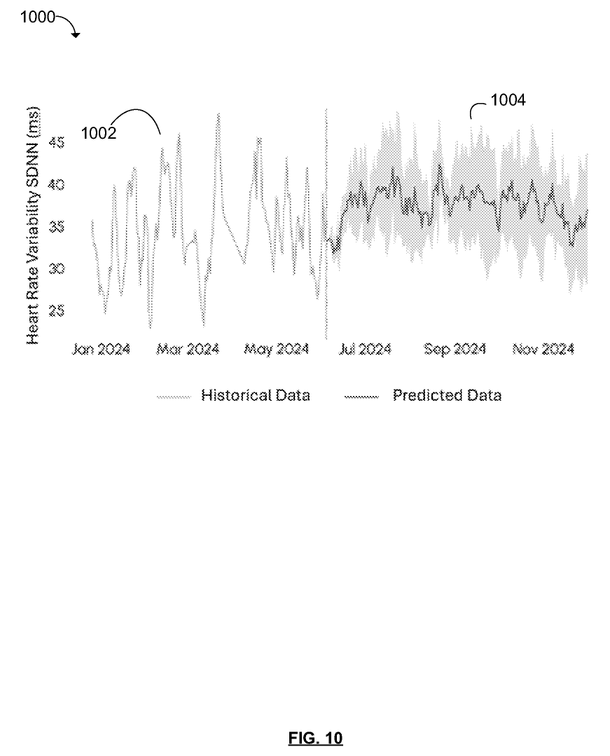

Referring next to FIG. 10, there is shown an example diagram 1000 of an individualized prediction algorithm that predicts a health outcome 1004 of a user in accordance with one or more embodiments. In this example, the individualized prediction algorithm applies a machine-learning model (e.g., a recurrent neural network) that is trained and validated on historical heart rate variability (HRV) data 1002 collected from a user’s wearable device (e.g., smart watch). The individualized prediction algorithm outputs predicted HRV data 1004 of the user over a defined period of time based on the historical HRV data 1002. The mean and confidence intervals are generated from bootstrapped iterations of the machine-learning model to reflect a similar variance to the historical HRV data 1002.

Referring next to FIG. 9, there is shown a method diagram 900 in accordance with one or more embodiments. The method diagram 900 provides a computer-implemented method for generating a health outcome prediction for a user.

At 902, user data associated with the user is received at a processor, the user data comprising passively collected data by a monitoring device in communication with the processor, the passive data collection monitoring device associated with the user.

At 904, clinical health outcome data is received at the processor, the clinical health outcome data comprising at least one health outcome based on clinical data associated with at least one individual.

At 906, a plurality of features from the user data are extracted at the processor.

At 908, the plurality of features and the clinical health outcome data are provided at the processor as inputs to at least one machine learning model to identify at least one selected feature from the plurality of features, the at least one selected feature having an associated predictive value to the clinical health outcome data for the user.

At 910, based on the at least one selected feature, an individualized prediction algorithm is generated at the processor for generating the health outcome prediction for the user, the individualized prediction algorithm being individualized to the user.

At 912, the individualized prediction algorithm is applied to the user data to generate the health outcome prediction of the user.

At 914 the health outcome prediction is output.

Optionally, the method may further comprise: prior to extracting, at the processor, the plurality of features from the user data, generating, at the processor, one or more features from the received user data, wherein the user data may comprise the received user data and the one or more features generated from the received user data; and generating, at the processor, one or more features from the received clinical health outcome data, wherein the clinical health outcome data may comprise the received clinical health outcome data and the one or more features generated from the received clinical health outcome data.

Optionally, the user data may further comprise self-reported health metric data, the self-reported health metric data comprising at least one user-provided answer to at least one prompt.

Optionally, the method may further comprise: providing, at a user input device in communication with the processor, the at least one prompt; and receiving, at the user input device, the at least one user-provided answer corresponding to the at least one prompt.

Optionally, the user input device comprises the passive data collection monitoring device.

Optionally, the at least one user-provided answer may provide data relating to at least one selected from the group of symptom data of the user, health event data of the user, symptom event data of the user, appointment data of the user, test result data of the user, medication data of the user and quality of life data of the user.

Optionally, the passive data collection monitoring device may comprise at least one selected from the group of a wearable device, a smartwatch, a smartphone, an accelerometer, a gyroscope, an electrocardiogram sensor, an electromyogram sensor, an arterial pulse sensor, a temperature sensor, a blood oxygen saturation sensor, a tissue carbon dioxide sensor, and an electrical impedance sensor.

Optionally, the clinical health outcome data may be received from at least one database associated with a healthcare database, system, or provider, or patient and/or disease registry.

Optionally, the at least one database may comprise data associated with a plurality of individuals, the data corresponding to at least one selected from the group of healthcare provider admissions data, surgery data, intervention data, diagnostic data, medication data, and specialist care data.

Optionally, the clinical health outcome data may further comprise data generated from the data of the at least one database, the generated data corresponding to at least one selected from the group of organ health data, organ function data, mental health data, cognitive function data, sleep data, mood data, pain data, sexual function data and mobility data.

Optionally, the generated data may be associated with the user.

Optionally, the plurality of features may comprise data corresponding to at least one selected from the group of physical activity data of the user, body composition data of the user, movement data of the user, sleep data of the user, organ health data of the user, organ function data of the user, sexual health data of the user, sexual function data of the user, mental health data of the user, cognitive function data of the user, productivity data of the user, mood data of the user, pain data of the user, symptoms data of the user, nutrition data of the user, medication data of the user, self-care data of the user, and wellness data of the user.

Optionally, identifying at least one selected feature from the plurality of features may comprise mapping, at the processor, the plurality of features and the clinical health outcome data.

Optionally, the at least one selected feature may be identified by applying a machine learning model to predict a health outcome.

Optionally, mapping, at the processor, the plurality of features and the clinical health outcome data may comprise performing, at the processor, at least one data modelling technique on the plurality of features and the clinical health outcome data.

Optionally, the at least one data modelling technique may be a dimensionality reduction based on the plurality of features and the clinical health outcome data.

Optionally, the generating the individualized prediction algorithm may comprise performing machine learning (e.g., dimensionality reduction) based on the at least one selected feature.

Optionally, performing the machine learning (e.g., dimensionality reduction) based on the at least on selected feature may comprise: determining a similarity metric for the at least one selected feature based on a desired clinical health outcome; and selecting at least one of the at least one selected feature based on the corresponding similarity metric.

Optionally, the method may further include receiving population-level data sets, processing the population-level data or sub-population-level data in the population-level data set, and wherein the extracting the plurality of features comprising extracting a subset of features from the population-level data or sub-population-level. For example, a small group of users may be clustered based on a population-based or sub-population-based similarity metric determined between those users. This population or sub-population based similarity metric may be calculated to cluster these users based on similarities in age, location, height, weight, injury profile, etc. The data from this “cluster” of similar users may be applied to generated “individualized” predictions equations that are single models applied to all individuals within the clustered subsample.

Optionally, the method may further comprise applying, at the processor, at least one goal function to the individualized prediction algorithm to improve goodness-of-fit of the individualized prediction algorithm.

Optionally, the applying, at the processor, the at least one goal function to the individualized prediction algorithm may occur iteratively.

Optionally, the at least one goal function may be at least one selected from the group of a mean squared error, an absolute value error, a root mean squared error value, an adjusted R2 value, an Akaike information criterion value, an absolute error value, a cross-entropy error value, a p-value based on a f-statistic value, or any other commonly used functions in model optimization.

Optionally, the root mean squared error value may be minimized.

Optionally, the outputting the health outcome prediction may comprise outputting the health outcome prediction at a user software application on a user device.

Optionally, the user device may comprise the passive data collection monitoring device.

Optionally, outputting the health outcome prediction may comprise outputting the health outcome prediction at a clinical software application on a clinical device.

The present invention has been described here by way of example only. Various modification and variations may be made to these exemplary embodiments without departing from the spirit and scope of the invention, which is limited only by the appended claims.

All publications, patents and patent applications are herein incorporated by reference in their entirety to the same extent as if each individual publication, patent or patent application was specifically and individually indicated to be incorporated by reference in its entirety.

We claim:

1. A computer-implemented method for generating a health outcome prediction for a user, the method comprising:
receiving, at a processor, user data associated with the user, the user data comprising monitoring data collected by a passive data collection monitoring device in communication with the processor, the passive data collection monitoring device associated with the user;
receiving, at the processor, clinical health outcome data, the clinical health outcome data comprising at least one health outcome based on clinical data associated with at least one individual;
extracting, at the processor, a plurality of features from the user data;
providing, at the processor, the plurality of features and the clinical health outcome data as inputs to at least one machine learning model to identify at least one selected feature from the plurality of features, the at least one selected feature having an associated predictive value to the clinical health outcome data for the user;
generating, at the processor, based on the at least one selected feature, an individualized prediction algorithm for generating the health outcome prediction for the user, the individualized prediction algorithm being individualized to the user;
applying, at the processor, the individualized prediction algorithm to the user data to generate the health outcome prediction of the user; and
outputting the health outcome prediction.

2. The computer-implemented method of claim 1, the method further comprising:
prior to extracting, at the processor, the plurality of features from the user data,
generating, at the processor, one or more features from the received user data, wherein the user data comprises the received user data and the one or more features generated from the received user data; and
generating, at the processor, one or more features from the received clinical health outcome data, wherein the clinical health outcome data comprises the received clinical health outcome data and the one or more features generated from the received clinical health outcome data.

3. The computer-implemented method of claim 1, wherein the user data further comprises self-reported health metric data, the self-reported health metric data comprising at least one user-provided answer to at least one prompt.

4. The computer-implemented method of claim 1, wherein the plurality of features comprises a plurality of variables corresponding to at least one selected from the group of physical activity data of the user, body composition data of the user, movement data of the user, sleep data of the user, organ health data of the user, organ function data of the user, sexual health data of the user, sexual function data of the user, mental health data of the user, cognitive function data of the user, productivity data of the user, mood data of the user, pain data of the user, symptoms data of the user, nutrition data of the user, medication data of the user, self-care data of the user, and wellness data of the user.

5. The computer-implemented method of claim 1, wherein identifying at least one selected feature from the plurality of features comprises mapping, at the processor, a plurality of variables and the clinical health outcome data.

6. The computer-implemented method of claim 5, wherein mapping, at the processor, the plurality of variables and the clinical health outcome data comprises performing, at the processor, at least one machine learning modelling technique on the plurality of variables and the clinical health outcome data.

7. The computer-implemented method of claim 6, wherein generating the individualized prediction algorithm comprises performing machine learning based on the at least one selected variable.

8. The computer-implemented method of claim 7, wherein implementing the machine learning model based on the at least one selected feature comprises:
determining a similarity metric for the at least one selected variable based on a desired clinical health outcome; and
selecting at least one of the at least one selected variable based on the corresponding similarity metric.

9. The computer-implemented method of claim 8, comprising:
providing, in the memory, a population-level data set;
extracting a set of population-level or sub-population features from the population-level data set at the processor; and
wherein the plurality of features comprises the extracted set of population-level or sub-population features.

10. A system for generating a health prediction outcome for a user, the system comprising:
a memory comprising:
at least one machine learning model;
a database comprising clinical health outcome data;
a processor in communication with the memory, the processor configured to:
provide, in the memory, user data associated with the user, the user data comprising passive data collection monitoring data, the monitor data collected by a passive data collection monitoring device in communication with the processor, the passive data collection monitoring device associated with the user;
receive, from the database, the clinical health outcome data, the clinical health outcome data comprising at least one health outcome based on clinical data associated with at least one individual;
extract a plurality of features from the user data;
provide the plurality of features and the clinical health outcome data as inputs to at least one machine learning model to identify at least one selected feature from the plurality of features, the at least one selected feature having an associated predictive value to the clinical health outcome data for the user;
generate, based on the at least one selected feature, an individualized prediction algorithm for generating the health outcome prediction for the user, the individualized prediction algorithm being individualized to the user;
apply the individualized prediction algorithm to the user data to generate the health outcome prediction of the user; and
output the health outcome prediction.

11. The system of claim 10, wherein the processor is further configured to:
prior to the processor being configured to extract a plurality of features from the user data,
generate one or more features from the provided user data, wherein the user data comprises the provided user data and the one or more features generated from the provided user data; and
generate one or more features from the received clinical health outcome data, wherein the clinical health outcome data comprises the received clinical health outcome data and the one or more features generated from the received clinical health outcome data.

12. The system of claim 10, the system further comprising a network interface in communication with the processor, the network interface configured to receive the clinical health outcome data from a second database associated with a healthcare provider.

13. The system of claim 10, wherein the processor being configured to identify at least one selected feature from the plurality of features comprises the processor being configured to map the plurality of variables and the clinical health outcome data.

14. The system of claim 13, wherein the processor being configured to map the plurality of variables and the clinical health outcome data comprises the processor being configured to perform at least one machine learning modelling technique on the plurality of variables and the clinical health outcome data.

15. The system of claim 10, wherein the processor being configured to generate the individualized prediction algorithm comprises the processor being configured to perform machine learning based on the at least one selected variable.

16. The system of claim 15, wherein the processor being configured to perform the dimensionality reduction based on the at least on selected variable comprises the processor being configured to:
determine a similarity metric for the at least one selected variable based on a desired clinical health outcome; and
select at least one of the at least one selected variable based on the corresponding similarity metric.

17. The system of claim 16, wherein the memory comprises:
providing, in the memory, a population-level data set;
and the processor is further configured to:
extract a set of population- or sub-population level features from the population-level data set; and
wherein the plurality of features comprises the extracted set of population-level or sub-population-level features.

18. The system of claim 10, wherein the processor is further configured to apply at least one goal function to the individualized prediction algorithm to improve goodness-of-fit of the individualized prediction algorithm.

19. The system of claim 18, wherein the processor is further configured to iteratively apply the at least one goal function to the individualized prediction algorithm.

20. The system of claim 10, wherein the processor is further configured to output the health outcome prediction at a clinical software application on a clinical device.
